(19) United States
(12) Patent Application Publication
(10) Pub. No.: US 2005/0084470 A1
(43) Pub. Date: Apr. 21, 2005

(54) Skin care and cleansing compositions containing oil seed product

(75) Inventor: Syed Husain Abbas, Seymour, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/685,866

(22) Filed: Oct. 15, 2003

(51) Int. Cl. A61K 7/075; A61K 7/08; A61K 7/06

(52) U.S. Cl. 424/70.22; 424/74

(57) ABSTRACT

Skin care and cleansing compositions are described that contain oil seed products that provide moisturization and in a preferred embodiment simultaneous exfoliation. Oil seed products contain oil bodies, seed shell and optionally other seed components. In a further embodiment, a toilet bar is provided that contains one or more anionic surfactants and 0.1 to 5.0% by wt. of oil seed product particles where a majority of the particles have a major axis length of between 0.01 to 200 microns.

### BACKGROUND OF THE INVENTION

1. Field of the Invention

The present invention relates to a skin care or cleansing composition suitable for topical application for moisturizing and cleansing the human body, such as the skin and hair. In particular, it relates to a toilet bar composition that is mild to the skin and which also moisturizes and, in a further embodiment exfoliates the skin.

2. The Related Art

Exfoliating toilet bars are well known. However, the majority of them are very irritating to the skin due to the fact that they are soap based, have high levels of harsh or marginally effective exfoliants, low levels of moisturizers, or some combination of the preceding. Certain prior art toilet bars with high levels of mild synthetic surfactants (“Syndet”) and high levels of moisturizers have been described as optionally having exfoliant particles or beads such as polyethylene beads, walnut shells, and apricot seeds, and the like. See e.g. U.S. Pat. No. 6,376,441; U.S. Pat. No. 6,342,470; U.S. Pat. No. 6,384,000; and U.S. Pat. No. 6,074,998.

Triglyceride oils derived from oil bearing plant seeds have been used in products such as creams, lotions, bar soaps, liquid soaps and shampoos as emollients and conditioners. High levels of oils can change the sensory behavior for the products. Occasionally it is difficult to use high levels of these oils in certain products, such as in toilet bars, since levels of oils greater than about 6 or 7% by wt. can create processing problems. Furthermore, oils may quickly get washed off due to solubilization in the micelles during the cleansing process. In an attempt to solve this problem, skin care and cleansing products have been formulated with liquid emollients encapsulated in beads. However, such beads can be destroyed during toilet bar processing, particularly under high pressure and temperatures. Surprisingly it has been found that formulating skin care and cleansing products as described below with oil seed product avoids such processing issues, especially for toilet bars, and such products having both one or more anionic surfactants and oilseed product can provide long lasting moisturization and in a preferred embodiment provide the user with both enhanced moisturization and exfoliation simultaneously. This causes the user's exfoliated skin to appear fresh and healthy as it removes the dull layer of dead skin, accompanied with deep cleansing leading to less clogged pores while at the same time moisturizing the skin to minimize irritation and dryness as shown by various art recognized techniques described below.

While not wishing to be bound by the following skin treatment theories, Applicants believe that exfoliation improves skin cleansing by helping to mechanically remove dirt and oil from the skin. Exfoliation also is believed to aid the process of desquamation. Desquamation is a natural process by which corneocytes are removed from the stratum corneum, which is the top layer of skin cells. Corneocytes are simply the cells that comprise the stratum corneum, and they are constantly being removed as the skin regenerates. Exfoliation aids in removing the flaky corneocytes that are ready to detach from the stratum corneum, and so promotes smoother, less flaky skin.

Other potential health benefits to exfoliation in addition to improved scale (flake) removal and oil removal, as suggested above, are reduction in bacteria on the skin, and increased blood flow to the skin due to the mechanical stimulation.

The inventive skin care and cleansing compositions under actual use conditions are expected to show improvements in skin softness, skin smoothness, and similar consumer perceived benefits such as exfoliation efficiency, mildness, moisturization efficiency, deposition efficiency, cleansing efficiency, and in a property such as skin abrasiveness, etc. based on changes from the baseline for these measurements using compositions without the inventive composition as quantified using the test methods described below.

The invention relates to the novel idea of the delivery of seed oils, and optionally other active and moisturizing components immobilized in the oil seed product, from a skin care or cleansing product by the use of oil seed product. In one respect the user can achieve the delivery of oil of the desired type in bars and liquid products. In another respect, the user can achieve exfoliation/massaging by varying the particle size and the quantity of the oil seed product.

### SUMMARY OF THE INVENTION

In one aspect the present invention is a skin care or cleansing composition, including but not limited to (a) about 2 to about 90% by wt. of an anionic surfactant; (b) about 0.1 to about 40% by wt. of an oil seed product wherein at least 50% by wt. of the product has a particle size range of about 0.01 to 200 microns; and (c) optionally a safe and effective amount of at least one active agent, moisturizing agent or blend thereof (as defined below).

Oil seed product includes native oil seeds, ground, broken or fractured oil seeds; oil seed cake, or a blend thereof. Oil seed product diameter is defined as the major axis of the particle.

In another aspect of the invention is a toilet bar, including but not limited to (a) about 0 to 30% by wt. of a fatty acid soap; (b) about 15 to 60% by wt. of a non-soap anionic surfactant; and (c) about 0.01 to 15% by wt. of an oil seed product having a particle size diameter of about 0.1 to 70 microns.

In a further aspect of the invention is a toilet bar, including but not limited to (a) about 30 to about 80% by wt. of a fatty acid soap; (b) about 5 to about 40% by wt. of a non-soap anionic surfactant; and (c) about 0.01 to 15% by wt. of an oil seed product having a particle size diameter of about 0.1 to 70 microns.

In a further aspect of the invention is a toilet bar, including but not limited to (a) about 30 to about 80% by wt. of a fatty acid soap; (b) about 0 to 10% by wt. of a non-soap anionic surfactant; and (c) about 0.01 to 15% by wt. of an oil seed product having a particle size diameter of about 0.1 to 70 microns.

### DETAILED DESCRIPTION OF THE INVENTION

In one aspect of the invention is a skin care or cleansing product, including but not limited to:
(a) about 2 to about 90% by wt. of an anionic surfactant;
(b) about 0.1 to about 40% by wt. of an oil seed product (where the minimum concentration in the inventive product is 0.01, 0.02, 0.05, 0.1, 0.2, 0.3, 0.4, or 0.5% by wt. and the maximum concentration is 1.0, 1.1, 1.5, 2, 3, 4, 5, 7, 9, 10, 11, 15, 20, 30 or 40% by wt.) wherein at least 50% by wt. of the product (preferably at least 60, 70, 80, 90 or 95%) has a particle size range of about 0.01 to about 200 microns (where more than 50, 60, 70, 80, 90, or 95% by wt. of the oil seed product has a minimum particle size of 0.01, 0.02, 0.05, 0.1, 0.2, 0.3, 0.4, 0.5, 1.0, 2, 5, 8, 10, 20, 30 or 40 microns respectively for nonexfoliating compositions or has a minimum particle size of 50, 60, 70, 80, 90, 100, 150 or 200 microns respectively for exfoliating compositions); and
(c) optionally a safe and effective amount of at least one active agent, moisturizing agent or blend thereof.

Advantageously the inventive product is selected from a toilet bar, a cream, a lotion, a shampoo, a liquid soap, an isotropic structured shower gel, or a crystalline structured body wash (e.g. having spherical micellar solution, rod micellar solution or liquid crystalline phases of lamellar, cubic, hexagonal structure and the like).

In another aspect of the invention is a toilet bar, including but not limited to:
(a) about 0 to 30% by wt. of a fatty acid soap (preferably 0 to about 15 or 20% by wt.);
(b) about 15 to 60% by wt. of a non-soap anionic surfactant; and
(c) about 0.01 to 15% by wt. of an oil seed product having a particle size diameter of about 0.1 to 70 microns.

Advantageously the inventive bar contains about 0 to 20% (preferably 15%) by wt. of a fatty acid soap and about 20 (preferably 25%) to 55% (preferably 50%); by wt. of a non-soap anionic surfactant. Preferably the bar further includes a safe and effective amount of at least one active agent, moisturizing agent or blend thereof. More preferably the oil seed product is selected from flax, soybean, sunflower, rapeseed, or palm and the like.

The fatty acid soaps in the inventive bar advantageously include a blend of C6 to C22 soaps (preferably a blend of C12 to C18 soaps) and a non-soap anionic surfactant selected from C8 to C14 acyl isethionates; C8 to C14 alkyl sulfates, C8 to C14 alkyl sulfosuccinates, C8 to C14 alkyl sulfonates; C8 to C14 fatty acid ester sulfonates, derivatives, or blends thereof.

In another aspect of the invention is a toilet bar, including but not limited to:
(a) about 30 to about 80% by wt. of a fatty acid soap;
(b) about 5 to about 40% by wt. of a non-soap anionic surfactant; and
(c) about 0.01 to 15% by wt. of an oil seed product having a particle size diameter of about 0.1 to 70 microns.

Advantageously this toilet contains about 40 or 50% to 60% or 70% by wt. of a fatty acid soap and about 7 or 10 to 20 or 30% by wt. of a non-soap anionic surfactant.

In a further aspect of the present invention is a toilet bar, including but not limited to:
(a) about 30 to about 80% by wt. of a fatty acid soap;
(b) about 0 to 10% by wt. of a non-soap anionic surfactant; and
(c) about 0.01 to 15% by wt. of an oil seed product having a particle size diameter of about 0.1 to 70 microns.

Advantageously this inventive toilet bar contains about 50 or 60 to 75% or 80% by wt. of a fatty acid soap and about 0, 0.1, 0.2, 0.5, 1.0, or 1.1 to 5 or 7% by wt. of a non-soap anionic surfactant.

In a preferred embodiment, by keeping the average particle size under 20 microns, advantageously in the range of 0.1-10 micron range, the delivery of the moisturizing components can be optimized from the oil seed product without exfoliation. By increasing the average particle size of the oil seed product to more than 20 or 25 microns, advantageously to more than 50 microns, the product provides both delivery of seed oils, and optionally other active and moisturizing components immobilized in the seed product, and desired exfoliation. Particle diameters above 50 microns acts as massaging components but the delivery of the seed oil and other active and moisturizing components is reduced because the oil seed particles will tend to wash away during the skin cleansing process. However for skin care or leave-on products that are not immediately washed away, the exfoliation and the delivery of the benefits may be achieved with oil seed particles with an average diameter of 150 to 200 microns. In addition, the level of oil in the oil seed product can be reduced by crushing the powder to obtain the desired amount of oil. In a further embodiment, the reduced oil and especially the oil free seed cake has been found to be an excellent exfoliant.

The oil seed product with desired properties of oil type, content and particle size can be added to conventional soap, combar, syndet bar and transparent soap bar formulations. Besides toilet bars the oil seed product can be used effectively in liquid products such as creams, lotions, liquid soaps, shower gels, body washes and shampoos. In the case of creams and lotions, the particles will remain in contact with the skin or hair for a longer period of time and are more efficient for the delivery of the oil bodies embedded in the oil seed product and optional acitve and moisturizing components immobilized in the seed product.

The inventive product under actual use conditions is expected to show improvements in skin softness, skin smoothness, and similar consumer perceived benefits such as exfoliation efficiency, mildness, moisturization efficiency, deposition efficiency, cleansing efficiency, and product property such as skin abrasiveness, etc. based on changes from the baseline for these measurements using products without the inventive composition as quantified using the test methods described below. These skin benefit parameters can also be expressed quantitatively as the ratio of the inventive product response to the comparative product response. Where the magnitude of the inventive product benefit improvement is expected to exceed the numerical result of the comparative bar, the observed ratio will be greater than 1.0; i.e. 1.02, 1.05, 1.07, 1.1, 1.2, 1.3, 1.4, 1.5, 1.6, 1.7, 1.8, 1.9, or 2.0. Where the magnitude of the inventive product benefit improvement is expected to fall below the numerical result of the comparative product, the observed ratio will be less than 1.0; i.e. 0.99, 0.98, 0.97, 0.95, 0.93, 0.90, 0.85, 0.80, 0.70, 0.60, 0.50, 0.40, 0.30, 0.20, or 0.10. Tables 1 to 3 below illustrate how various properties of the inventive product, in this case a toilet bar, are expected to compare to four different comparative bars. The test methods that may be used to measure the properties are provided below.

TABLE 1
Inventive bar vs. Comparative A⁽¹⁾ (Soap bar
without oil seed product)

| Property | Ratio vs. Comparative A |
|---|---|
| Mildness | >1 |
| Moisturization | >1 |
| Moisturizer deposition | >1 |
| Softness | >1 |
| Smoothness | >1 |
| Skin abrasiveness⁽²⁾ | <1 |

⁽¹⁾Comparative A: True Blue Spa: Look Cool Minty Fresh Buffing Bar ® from Bath & Body Works containing: Sodium Palmitate, Sodium Cocoate, Water, Glycerin, Fragrance, Jojoba Wax, Pentasodium Pentetate, Tetrasodium etidronate, Gaultheria Procumbens (Wintergreen) Leaf Oil, Menta Piperita (Peppermint) Leaf Oil, TitaniumDioxide (CI77891), Ultramarines (CI 77007)
⁽²⁾Higher value means worse.

TABLE 2
Inventive bar Vs Comparative B⁽³⁾ (Syndet
bar without oil seed product)

| Property | Ratio vs. Comparative B |
|---|---|
| Exfoliation | >1 |
| Cleansing efficiency | >1 |
| Moisturizer deposition | =>1 |
| Softness | =>1 |
| Smoothness | =>1 |
| Bar sensory exfoliation | =>1 |

⁽³⁾E.g. Dove ® Bar available from Unilever which contains Sodium Cocyl Isethionate, Stearic Acid, Sodium Tallowate, Water, Sodium Isethionate, Coconut Acid, Sodium Stearate, Cocamidopropyl Betaine, Sodium Cocoate, Fragrance, Sodium Chloride, Titanium Dioxide, Tetrasodium EDTA, Trisodium Etidronate, and BHT as indicated on the label.

TABLE 3
Inventive bar vs. Comparative C⁽⁴⁾ (Syndet bar with
exfoliants other than oil seed product)

| Property | Ratio vs. Comparative C |
|---|---|
| Exfoliation⁽⁵⁾ | >1 |
| Cleansing efficiency | >1 |
| Moisturizer deposition | =>1 |
| Softness⁽⁵⁾ | =>1 |
| Smoothness⁽⁵⁾ | =>1 |
| Bar sensory exfoliation | =>1 |

⁽⁴⁾E.g. Dove ® Bar available from Unilever which contains Sodium Cocyl Isethionate, Stearic Acid, Sodium Tallowate, Water, Sodium Isethionate, Coconut Acid, Sodium Stearate, Cocamidopropyl Betaine, Sodium Cocoate, Fragrance, Sodium Chloride, Titanium Dioxide, Tetrasodium EDTA, Trisodium Etidronate, and BHT as indicated on the label with added polyethylene beads.

Oil Seed Products

Among the oil seed products useful herein are those seed products obtainable from plant species such as almond (Prunus dulcis); anise (Pimpinella anisum); avocado (Persea spp.); beach nut (Fagus sylvatica); borage (also known as evening primrose) (Boragio officinalis); Brazil nut (Bertholletia excelsa); candle nut (Aleuritis tiglium); carapa (Carapa guineensis); cashew nut (Ancardium occidentale); castor (Ricinus communis); coconut (Cocus nucifera); coriander (Coriandrum sativum); cottonseed (Gossypium spp.); crambe (Crambe abyssinica); Crepis alpina; croton (Croton tiglium); Cuphea spp.; dill (Anethum gravealis); Euphorbia lagascae; Dimorphoteca pluvialis; false flax (Camolina sativa); fennel (Foeniculum vulgaris); groundnut (Arachis hypogaea); hazelnut (coryllus avellana); hemp (Cannabis sativa); honesty plant (Lunnaria annua); jojoba (Simmondsia chinensis); kapok fruit (Ceiba pentandra); kukui nut (Aleuritis moluccana); Lesquerella spp., linseed/flax (Linum usitatissimum); macademia nut (Macademia spp.); maize (Zea mays); meadow foam (Limnanthes alba); mustard (Brassica spp. and Sinapis alba); oil palm (Elaeis guineeis); oiticia (Licania rigida); paw paw (Assimina triloba); pecan (Juglandaceae spp.); perilla (Perilla frutescens); physic nut (Gatropha curcas); pilinut (Canarium ovatum); pine nut (pine spp.); pistachio (Pistachia vera); pongam (Bongamin glabra); poppy seed (Papaver soniferum); rapeseed (Brassica spp.); safflower (Carthamus tinctorius); sesame seed (Sesamum indicum); soybean (Glycine max); squash (Cucurbita maxima); sal tree (Shorea rubusha); Stokes aster (Stokesia laevis); sunflower (Helianthus annuus); tukuma (Astocarya spp.); tung nut (Aleuritis cordata); vernonia (Vernonia galamensis); and mixtures thereof, and the like.

More preferred for use herein are oil seed products obtained from the following plant species: Brazil nut (Bertholletia excelsa); castor (Ricinus communis); coconut (Cocus nucifera); coriander (Coriandrum sativum); cottonseed (Gossypium spp.); groundnut (Arachis hypogaea); jojoba (Simmondsia chinensis); linseed/flax (Linum usitatissimum); maize (Zea mays); mustard (Brassica spp. and Sinapis alba); oil palm (Elaeis guineeis); olive (Olea europaea); rapeseed (Brassica spp.); safflower (Carthamus tinctorius); soybean (Glycine max); squash (Cucurbita maxima); sunflower (Helianthus annuus); and mixtures thereof.

Oil bodies are defined as the naturally occurring water insoluble oil fraction stored in the seed. Oil bodies include the powdered particulate having triacylglycerides, and may also include any of the following: phospholipids, associated proteins and blends thereof. Useful oil seed product for this invention contains oil bodies having a minimum concentration range of about 0.01, 0.02, 0.05, 0.1, 0.2, 0.3, 0.5, 1.0 or 1.5% by wt. to about 2, 5, 10, 20, 30, 40 or 50% by wt. or more based on the dry oil seed product. The oil content of the seeds may be varied according to the desired application of the product by the addition or removal of native oil, or by the substitution of a quantity of different seed oil and/or another emollient as defined below.

Surfactants:

Surfactants are an essential component of the inventive toilet skin care or cleansing composition. They are compounds that have hydrophobic and hydrophilic portions that act to reduce the surface tension of the aqueous solutions they are dissolved in. Useful surfactants can include anionic, nonionic, amphoteric, and cationic surfactants, and blends thereof.

Anionic Surfactants:

Synthetic Anionic Surfactants

The cleansing composition of the present invention contains one or more non-soap anionic detergents (syndets). Preferably the syndets have a zein value of 50 or less. Zein value may be measured using the test method described below.

The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of greater than 1.0, preferably greater than 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:
R⁴O₂CCH₂CH(SO₃M)CO₂M; and
amide-MEA sulfosuccinates of the formula;
   R⁴CONHCH₂CH₂O₂CCH₂CH(SO₃M)CO₂M
wherein R⁴ ranges from C₈-C₂₂ alkyl and M is a solubilizing cation.

Sarcosinates are generally indicated by the formula:
R¹CON(CH₃)CH₂CO₂M,
wherein R¹ ranges from C₈-C₂₀ alkyl and M is a solubilizing cation.

Taurates are generally identified by formula:
R²CONR³CH₂CH₂SO₃M
wherein R² ranges from C₈-C₂₀ alkyl, R³ ranges from C₁-C₄ alkyl and M is a solubilizing cation.

The inventive skin care or cleansing composition preferably contains C₈-C₁₄ acyl isethionates. These esters are prepared by reaction between alkali metal isethionate with mixed aliphatic fatty acids having from 6 to 12 carbon atoms and an iodine value of less than 20.

The acyl isethionate may be an alkoxylated isethionate such as is described in Ilardi et al., U.S. Pat. No. 5,393,466, titled “Fatty Acid Esters of Polyalkoxylated isethonic acid; issued Feb. 28, 1995; hereby incorporated by reference. This compound has the general formula: 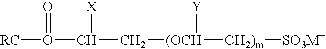
wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

In another embodiment of the inventive toilet bar, there is less than 5% by wt. of any of the following anionic surfactants: alkyl sulfates, alkyl sulfonates, alkyl benzene sulfonates, alkyl alkoxy sulfates, acyl taurides, acyl sulfates, and polyhydroxy fatty acid amides either individually or of a blend thereof. Preferably there is less than 1%, and more preferably less than 0.1% by wt. of these surfactants

Soaps.

The inventive skin care or cleansing product may contain soap, The term “soap” is used herein in its popular sense, i.e., the alkali metal or alkanol ammonium salts of alkane- or alkene monocarboxylic acids. Sodium, potassium, mono-, di- and tri-ethanol ammonium cations, or combinations thereof, are suitable for purposes of this invention. In general, sodium, potassium, ammonium, mono-, di-, and tri-ethanol soaps of saturated C₈-C₁₄ alkyl chains and unsaturated fatty acids, preferably having C₈-C₂₂ alkyl chains, are soluble soaps.

Amphoteric Surfactants

One or more amphoteric surfactants may be used in this invention. Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula: 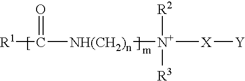
where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric surfactants within the above general formula include simple betaines of formula: 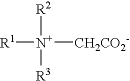
and amido betaines of formula: 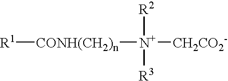
where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula: 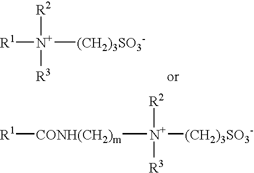
where m is 2 or 3, or variants of these in which —(CH₂)₃ SO₃⁻ is replaced by

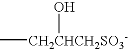

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Nonionic Surfactants

One or more nonionic surfactants may also be used in the skin care or cleansing composition of the present invention.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Cationic Skin Conditioning Agents

An optional component in compositions according to the invention is a cationic skin feel agent or polymer, such as for example cationic celluloses. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200, and quaternary ammonium compounds such as alkyldimethylammonium halogenides.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation.

Other preferred cationic compounds that are useful in the present invention include amido quaternary ammonium compounds such as quaternary ammonium propionate and lactate salts, and quaternary ammonium hydrolyzates of silk or wheat protein, and the like. Many of these compounds can be obtained as the Mackine™ Amido Functional Amines, Mackalene™ Amido functional Tertiary Amine Salts, and Mackpro® cationic protein hydrolysates from the McIntyre Group Ltd. (University Park, Ill.).

In a preferred embodiment of the invention having a hydrolyzed protein conditioning agent, the average molecular weight of the hydrolyzed protein is preferably about 2500. Preferably 90% of the hydrolyzed protein is between a molecular weight of about 1500 to about 3500. In a preferred embodiment, MACKPRO™ WWP (i.e. wheat germ amido dimethylamine hydrolyzed wheat protein) is added at a concentration of 0.1% (as is) in the bar. This results in a MACKPRO™ WWP “solids” of 0.035% in the final bar formula for this embodiment.

Cationic Surfactants

One or more cationic surfactants may also be used in the inventive self-foaming cleansing composition.

Examples of cationic detergents are the quaternary ammonium compounds such as alkyldimethylammonium halogenides.

Other suitable surfactants which may be used are described in U.S. Pat. No. 3,723,325 to Parran Jr. titled “Detergent Compositions Containing Particle Deposition Enhancing Agents” issued Mar. 27, 1973; and “Surface Active Agents and Detergents” (Vol. I & II) by Schwartz, Perry & Berch, both of which are also incorporated into the subject application by reference.

In addition, the inventive composition, especially the toilet bar of the invention may include 0 to 15% by wt. optional ingredients as follows:
perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer) and the like; all of which are useful in enhancing the appearance or cosmetic properties of the product.

The compositions may further comprise preservatives such as dimethyloldimethylhydantoin (Glydant XL1000), parabens, sorbic acid etc., and the like.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) and the like may be used advantageously in amounts of about 0.01% or higher if appropriate.

In addition to the oil seed product, moisturizers also expressed as either humectants and emollients may be advantageously used in the present invention. Humectants such as polyhydric alcohols, e.g. glycerin and propylene glycol, and the like; and polyols such as the polyethylene glycols listed below and the like may be used. Humectants may be employed at levels greater than 0.01, 0.05, 0.1, 0.2, 0.5, 0.9, 1.0, 1.1, 2.0, 3.0, 5, 9, 10, 11, 15 or 20% by wt.

| | |
|---|---|
| Polyox WSR-205 | PEG 14M, |
| Polyox WSR-N-60K | PEG 45M, or |
| Polyox WSR-N-750 | PEG 7M. |

Emollients may be added to the inventive product separately from the oil seed product or all or a portion of the oil seed product may be used that has been enriched in emollients. These separate emollients that are distinct from the oil seed product may be composed of a single moisturizing agent component or they may be a mixture of two or more compounds one or all of which may have a moisturizing aspect. In addition, the moisturizing agent itself may act as a carrier for other components one may wish to add to the inventive skin care or cleansing product.

Hydrophobic emollients, hydrophilic emollients, or a blend thereof may be used. Preferably, hydrophobic emollients are used in excess of hydrophilic emollients in the inventive toilet skin care or cleansing composition. Hydrophobic emollients including those native to and releasably contained in the oil seed product are preferably present in a concentration greater than about 0.01, 0.05, 0.1, 0.2, 0.5, 0.9, 1.0, 1.1, 2.0, 3.0, 5, 9, 10, 11, 15, 20, 25 or 30% by wt. Hydrophobic emollients added separately from the oil seed product may be present in the inventive product at levels greater than about 0.01, 0.05, 0.1, 0.2, 0.5, 0.9, 1.0, 1.1, 2.0, 3.0, 5, 9, 10, 11, 15, or 20% by wt. The term “emollient” is defined as a substance which softens or improves the elasticity, appearance, and youthfulness of the skin (stratum corneum) by increasing its water content, and keeps it soft by retarding the decrease of its water content.

Useful emollients include the following:
(a) silicone oils and modifications thereof such as linear and cyclic polydimethylsiloxanes; amino, alkyl, alkylaryl, and aryl silicone oils;
(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;
(c) waxes such as carnauba, spermaceti, beeswax, lanolin, and derivatives thereof;
(d) hydrophobic and hydrophilic plant extracts;
(e) hydrocarbons such as liquid paraffin, vaseline, microcrystalline wax, ceresin, squalene, pristan and mineral oil;
(f) higher fatty acids such as lauric, myristic, palmitic, stearic, behenic, oleic, linoleic, linolenic, lanolic, isostearic, arachidonic and poly unsaturated fatty acids (PUFA);
(g) higher alcohols such as lauryl, cetyl, stearyl, oleyl, behenyl, cholesterol and 2-hexydecanol alcohol;
(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;
(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils; and
(j) mixtures of any of the foregoing components, and the like.

Preferred emollient moisturizing agents are selected from fatty acids, triglyceride oils, mineral oils, petrolatum, and mixtures thereof. Further preferred emollients are fatty acids.

Exfoliants

The inventive product, especially the inventive bar may contain oil seed product particles that are greater than 50 microns that help remove dry skin. Not being bound by theory, the degree of exfoliation depends on the size and morphology of the particles. Large and rough particles are usually very harsh and irritating. Very small particles may not serve as effective exfoliants.

Additional exfoliants other than oil seed particles may be used. Such exfoliants used in the art include natural minerals such as silica, talc, calcite, pumice, tricalcium phosphate; seeds such as rice, apricot seeds, etc; crushed shells such as almond and walnut shells; oatmeal; polymers such as polyethylene and polypropylene beads, flower petals and leaves; microcrystalline wax beads; jojoba ester beads, and the like. These exfoliants come in a variety of particle sizes and morphology ranging from micron sized to a few mm. They also have a range of hardness. Some examples are given in table 4 below.

TABLE 4

| Material | Hardness (Mohs) |
|---|---|
| Talc | 1 |
| Calcite | 3 |
| Pumice | 4-6 |
| Walnut Shells | 3-4 |
| Dolomite | 4 |
| Polyethylene | ˜1 |

Optional Active Agents

Advantageously, active agents other than moisturizers defined above may be added to the oil seed product in a safe and effective amount before or during formulation of the inventive skin care and cleansing products in order to immobilize and/or stabilize such ingredients so as to allow more controlled release and effective utilization of these ingredients to and by the skin during the use of the product. These active ingredients may be advantageously selected from bactericides, vitamins, anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; or mixtures thereof; and the like.

These active agents may be selected from water soluble active agents, oil soluble active agents, pharmaceutically-acceptable salts and mixtures thereof. Advantageously the agents will be soluble or dispersible in the oil bodies present in the oil seed product. The term “active agent” as used herein, means personal care actives which can be used to deliver a benefit to the skin and/or hair and which generally are not used to confer a conditioning benefit, as hereinafter defined. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors. Preferably the compositions of the present invention comprise from about 0.01% to about 50%, more preferably from about 0.05% to about 25%, even more preferably 0.1% to about 10%, and most preferably 0.1% % to about 5%, by weight of the active agent component.

A wide variety of active agent ingredients are useful herein and include those selected from anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sunscreen actives, sebum stimulators, sebum inhibitors, anti-glycation actives and mixtures thereof and the like.

Anti-acne actives can be effective in treating acne vulgaris, a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides as described in European Patent Specification No. 556,957; ascorbic acid; biotin; biotin esters; phospholipids, mixtures thereof, and the like.

Non-steroidal Cosmetic Soothing Actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like. Many of these cosmetic soothing actives are described in U.S. Pat. No. 4,985,459 to Sunshine et al., issued Jan. 15, 1991, incorporated by reference herein in its entirety.

Artificial tanning actives can help in simulating a natural suntan by increasing melanin in the skin or by producing the appearance of increased melanin in the skin. Nonlimiting examples of artificial tanning agents and accelerators include dihydroxyacetaone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycin namate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Active ingredients in the present invention may also include anti-itch ingredients. Suitable examples of anti-itch ingredients which are useful in the compositions of the present invention include hydrocortisone, methdilizine and trimeprazineare, mixtures thereof, and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like.

A nonlimiting example of an anti-glycation agent which is useful in the compositions of the present invention would be Amadorine (available from Barnet Products Distributor), and the like.

Active agents may be immobilized within or adjacent to the oil seed product by any art recognized technique such as by contacting the oil seed product with a solution or suspension (i.e. a transfer medium) of one or more active agents under conditions sufficient to allow the transfer of a desired quantity of active agent to the oil seed or oil bodies contained in the oil seed product. This step may then followed by the isolation of the enriched oil seed product via filtration, evaporation of the solvent or any other art recognized process step to yield oil seed product containing active agent. Alternatively the enriched oil seed product may be directly formulated into a skin care or cleansing product without being isolated from the transfer medium provided the medium is compatible with the formulation to which it is added, or a combination of enriched oil seed product and transfer medium may be used. The transfer medium may be a liquid, gel, solid, particulate or blend thereof, and the like.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated. Physical test methods are described below:

The following inventive toilet bars may be formulated according to the manufacturing methods described below:

### EXAMPLE 1

### Toilet Soaps

The Seed powder can be added in chip mixer along with soap base (described below), fragrance and other minor components. The soap base (see table 5) can be of varying ratios of the different fatty acid soap components. The minor components can be the common ingredients used in soap bar manufacture including emollients, antibacterial agents, colorants, opacifiers, brighteners etc. The mixed mass is then milled/refined and plodded. The plodded bars are then pressed into the desired shape. Four examples of such bars (a, b, c and d) may be made as follows:

Bars

| Ingredients | a | b | c | d |
|---|---|---|---|---|
| Soap Base | 87.9 | 86.8 | 87.5 | 87.5 |
| Perfume | 1.0 | 1.0 | 1.0 | 1.0 |
| Irgason DP 300 | — | 0.2 | — | — |
| Petrolatum | — | 1.0 | — | 0.5 |
| Mineral oil | — | — | 0.5 | — |
| Wheat Protein | 0.1 | — | — | — |
| Titanium Dioxide | 0.5 | 0.5 | 0.5 | 0.5 |
| Flax Seed Powder | 0.5 | 0.5 | 0.5 | 0.5 |
| Water | 10.0 | 10.0 | 10.0 | 10.0 |
| Total | 100 | 100 | 100 | 100 |

### EXAMPLE 2

### Combar

The oil seed powder can be added in chip mixer along with the combar base (described below), having a mixture of soapy and non-soapy actives and other minor components. The mixed mass is then milled/refined and plodded. The plodded bars are pressed into the desired shape. Three examples of such bars e, f, and g may be made as follows:

| Ingredients | e | f | g |
|---|---|---|---|
| Soap Base | 77.8 | 84.8 | 72.5 |
| Sodium Cocoyl Isethionate | 10.0 | — | 10.0 |
| Alfa Olefin Sulfonate | — | 3.0 | 3.0 |
| Cocoamidopropyl Betaine | — | — | 2.0 |
| Perfume | 1.0 | 1.0 | 1.0 |
| Salt (sodium chloride) | 0.7 | 0.7 | 1.0 |
| Flax Seed Powder | 0.5 | 0.5 | 0.5 |
| Water | 10.0 | 10.0 | 10.0 |
| Total | 100 | 100 | 100 |

### EXAMPLE 3

### Syndet Bar

The oil seed powder can be added in chip mixer along with s syndet base (described below), having a mixture of non-soapy actives and the other minor components. A small quantity of soap base can be added in chip mixer to facilitate the process. The mixed mass is then milled/refined and plodded.

The plodded bars are pressed into the desired shape. The base is made by melting sodium cocoyl isethionate with stearic acid, salt and small quantity of soap (see table 5). This molten mass is chill roll milled and then used as base. Three examples of such bars (h, i and j) may be made as follows:

| Ingredients | h | i | j |
|---|---|---|---|
| Base | 93.5 | 93.0 | 90.5 |
| Perfume | 1.0 | 1.0 | 1.0 |
| Flax Seed Powder | 0.5 | 0.5 | 0.5 |
| Petrolatum | — | 0.5 | — |
| Sunflower Seed Oil | — | — | 3.0 |
| Water | 5.0 | 5.0 | 5.0 |
| Total | 100 | 100 | 100 |

### EXAMPLE 4

### Translucent Bar

First translucency is created in the transparent soap base (described below), by high shear mixing at 47-50 C for about 30 minutes to 50 minutes. The powder is added in the mixer along with fragrance at the end of this 30 to 50 minute period and blended for about 5 minutes. The mass is then plodded under controlled conditions of temperature to get a translucent bar with the dispersed particulate of the seed powder. Four examples of such bars (k, l, m and n) may be made as follows:

| Ingredients | k | l | m | n |
|---|---|---|---|---|
| 82/18 Soap¹ (anhydrous) | 69.5 | 71.5 | 72.5 | 72.5 |
| Glycerin | 5.0 | 3.0 | 5.0 | — |
| Propylene Glycol | 5.0 | 3.0 | — | 5.0 |
| Triethanolamine | — | 3.0 | 3.0 | 3.0 |
| Perfume | 1.0 | 1.0 | 1.0 | 1.0 |
| Flax Seed Powder | 0.5 | 0.5 | 0.5 | 0.5 |
| Water | 18.0 | 18.0 | 18.0 | 18.0 |
| Total | 100 | 100 | 100 | 100 |

¹Tallowate/Cocoate

TABLE 5
Toilet bar bases:

| Soap Base | Syndet Base |
|---|---|
| 50-90% Tallow Soap | 40-60% SCI |
| 10-50% Coco Soap | 2-8% Alfa Olefin Sulfonate |
| 200-500 ppm EDTA | 15-40% Stearic Acid |
| 0.1-1.5% Salt | 0-15% Tallowate/Cocoate Soap |

### EXAMPLE 5

### Liquid Soap

The liquid soap is made by mixing of the ingredients at 25 C or by dissolving the less soluble components at elevated temperature typically in the range of 65-70° C. Before mixing in the oil seed product the temperature is brought back to 25° C. The powder is added in the blend and mixed gently to avoid any aeration. The viscosity of the liquid soap desired for the product is adjusted by using common salt. Higher particle size of the oil seed product needs higher viscosity to get a stable non separating suspension of the powder. Three examples of such bars (o, p and q) may be made as follows:

| Ingredients | o | p | q |
|---|---|---|---|
| Sodium Lauryl Ether Sulfate | 16.0 | 13.0 | 10.0 |
| Cocoamidopropyl Betaine | 3.0 | 3.0 | 5.0 |
| Ethylene Glycol Distearate | — | 1.0 | 1.0 |
| Salt | 0.4 | 0.5 | 0.2 |
| Perfume | 1.0 | 1.0 | 1.0 |
| Flax Seed Powder | 0.5 | 0.5 | 0.5 |
| Water | 79.1 | 81.0 | 82.5 |
| Total | 100 | 100 | 100 |

### EXAMPLE 6

### Shampoos

The powder should be added in the shampoo formulation near room temperature and mixed gently. The procedure of making shampoo is same as described for liquid soap. Four inventive shampoo formulations are described below:

| Ingredients | r | s | t |
|---|---|---|---|
| Sodium Lauryl Ether Sulfate | 16.0 | 13.0 | 10.0 |
| Cocoamidopropyl Betaine | 3.0 | 6.0 | 5.0 |
| Ethylene Glycol Distearate | — | 1.0 | 1.0 |
| Polymer JR400 | 0.5 | 0.3 | — |
| Cationic Guar | 0.2 | 0.3 | 0.8 |
| Dimethicone | 3.0 | — | 2.0 |
| Salt | 0.4 | 0.5 | 0.2 |
| Perfume | 1.0 | 1.0 | 1.0 |
| Flax Seed Powder | 0.5 | 0.5 | 0.5 |
| Water | q.s. to 100% | q.s. to 100% | q.s. to 100% |

### EXAMPLE 7

### Melt Cast Bar Formulations

Melt cast bars may also be made depending on the melt properties of the particular blend used. In this case, the formulation is made by melting all the ingredients at 60-95° C. The homogeneous liquid transparent/opaque is cooled to preferably at 65-70°. The powder and fragrance is added at this temperature in the melt, homogenized and then product is poured into molds. The blend is then allowed to solidify under ambient or accelerated cooling conditions. It is preferred to premix the oil seed product in glycerin/water or any other suitable liquid for better dispersion. The bars after cooling can be transparent, translucent or opaque depending upon the formulation. Use of hydrophobic moisturizing components such as triglycerides in transparent soap formulations may reduce the transparency. The oil seed powder advantageously holds the oil in it, thereby preserving the transparency and providing an attractive visual effect in the bar. Control of particle size and the viscosity of the melt are critical for stable suspension of the powder. Three examples of opaque melt cast formulations (u, v and w) are as follows:

| Ingredients | u | v | w |
|---|---|---|---|
| Sodium Cocoyl Isethionate | 40.77 | 42.6 | 42.00 |
| Stearic acid/Palmitic Acid | 10.00 | 10.00 | 10.00 |
| Coco Fatty acid | 0.67 | — | — |
| Sodium Isethionate | 0.56 | — | — |
| Alfa Olefin Sulfonate | 5.00 | 3.70 | 4.00 |
| Sodium Lauryl Ether Sulfate (2 EO) | 5.00 | 3.7 | 1.00 |
| Perfume | 1.00 | 1.00 | 1.00 |
| Titanium Dioxide | 1.00 | 1.00 | 1.00 |
| Propylene Glycol | 7.00 | 5.00 | 5.00 |
| Flax Seed Powder | 5.00 | 7.00 | 5.00 |
| Glycerin | 4.00 | 4.00 | 6.00 |
| Lauryl Alcohol | 5.00 | 5.00 | 6.00 |
| 12-hydroxystearic acid | 11.00 | 13.00 | 15.00 |
| Water | q.s. to | q.s. to | q.s. to 100% |
| | 100% | 100% | |

### EXAMPLE 8

### Skin Creams and Lotions

The exfoliation and the delivery of oil as moisturizing component in leave on products such as lotions and creams can be achieved by using oil seed powder advantageously at the level of up to 15% in the formulation. The particle is preferably in the range of about 25-50 microns. Two formulations (x & y) of inventive skin creams are given below:

| Ingredients | x | y |
|---|---|---|
| Oil phase | | |
| Stearic Acid | 12.0 | 15.0 |
| Palmitic acid | 3.0 | — |
| Isopropyl Myristate | 2.8 | 3.00 |
| Water Phase | | |
| Potassium Hydroxide | 0.5 | 0.50 |
| Triethanolamine | 0.3 | 0.50 |
| Glycerin | 5.00 | 5.00 |
| Water | 69.3 | 73.9 |
| Mustard Seed Powder | 5.00 | — |
| Perfume | 0.30 | 0.30 |
| Flax Seed Powder | — | 2.00 |
| Preservative | 0.10 | 0.10 |

The oil phase is first blended and melted at 75 C. The aqueous phase is also heated to 75 C. The two phases are mixed slowly with continuous homogenization and the temperature is brought down to about 40 C. Fragrance, Seed Powder and the preservatives are added and mixed gently to avoid any aeration.

Description of Test Methods

Methods of Testing

One or more of the following tests can be used to characterize the inventive skin care or cleansing product, especially toilet bars and compare it to comparative toilet bars.

a) Exfoliation Test:

A suitable corneocyte staining dye (e.g.: gentian violet) is applied to a 2-5 cm diameter spot on skin (arm/leg or any other part of the body as desired) and left on for 5 minutes to ensure uniform staining of the skin surface cells (corneocytes). The excess dye is then washed away by rinsing the spot under running water at 35 C for 30 sec. with no rubbing of the skin.

The stained sites are then washed with the test product. For the bar the following wash method is adopted. Wet the spot on skin, pre-wet the bar, rub bar directly on spot for 30 sec (back and forth motion), rinse for 15 sec under running water at 35 C for 30 sec. with no rubbing of the skin and gently pat dry. Allow the site to dry for 10 minutes. A d-squame tape (Cuderm® manufactured by CuDerm Corporation, (Dallas, Tex.) is applied on the washed spot under a uniform pressure for 30 sec. and then removed. The d-squame tape is imaged using a Kodak DCS 420 digital camera with a 105 mm lens. The image is analyzed using Optimas image analysis software for area covered/total intensity of stained cells (Optimas® is available from Media Cybernetics, Silver Springs, Md.). By comparing this data to similar information from an unwashed site, one can estimate the amount of exfoliation caused by the test product as follows:
Exfoliation=(area of d-squame covered by stain on unwashed site−area of d-squame covered by stain on washed site)/(area of d-squame covered by stain on unwashed site)

Alternately exfoliation can also be evaluated in a consumer test as follows:

The test protocol consists of
1) Recruiting approx. 10-20 women in the age group of 25-65 and who are
complexion bar users.
2) Use test and comparative products for a week each. Half the panelists
would use the test product first and the other half would use the comparative product first.
3) At the end of the test, the panelists rate their preference (on a 0-5 point scale) on the attribute of “exfoliation”.

The degree of exfoliation is defined as the consumer rating on the 0-5 point scale

b) Mildness Test:

i) Forearm Controlled Application Test (Fcat) Clinical Test Methodology

This controlled washing test is similar to that described by Ertel et al (A forearm controlled application technique for estimating the relative mildness of personal cleansing products, J. Soc. Cosmet. Chem., 46, 67 (1995)).

Subjects report to the testing facility for the conditioning phase of the study, which consists of using an assigned marketed personal washing cleanser for general use at home, up to four days prior to start of the product application phase. On Day 1 of the product application phase, a visual assessment is made to determine subject qualification. Subjects must have dryness scores >1.0 and erythema scores >0.5, and be free of cuts and abrasions on or near the test sites to be included in the product application phase. Subjects who qualify to enter the product application phase will then be instructed to discontinue the use of the conditioning product and any other skin care products on their inner forearms, with the exception of the skin cleansing test formulations that are applied during the wash sessions.

Qualified subjects will then have four 3.0-cm diameter (round) evaluation sites marked on each of the forearms using a skin safe pen (a total of eight sites). Visual evaluations for erythema and dryness will be conducted immediately prior to the first wash in each session and again in the afternoon of the final day (Day 5).

Washing Procedure for Bar Products

1. Both arms are washed simultaneously. Test sites are treated in a sequential manner starting with the site closest to the flex area, ending with the site proximal to the wrist.

2. The sites closest to the flex area of the inner forearm of both the right and left arm are moistened with warm water (90°-100° F.).

3. A moistened Masslinn towel is rubbed in a circular motion on a wetted test bar for approximately 6 seconds by study personnel which will result in 0.2-0.5 g of product to be dispensed.

4. The site is washed with the designated product for 10 seconds followed by a 90-second lather retention phase.

5. The above procedure (1-4) is then repeated for each of the test sites. Sites are then be rinsed (e.g. using a temperature of 35 C) for fifteen seconds and patted dry.

6. Upon completion the entire procedure is repeated (two washes/session).

For Liquid Products: A technician will prepare liquid products just prior to the wash session by dispensing between 0.1 g and 0.5 g of product either directly onto the skin or a moistened Maslinn towel or alternative application material. The washing procedure outlined above will then be used.

Evaluation Methods

Baseline visual assessments are made prior to the start of the product application phase, and immediately before each wash session thereafter, to evaluate dryness and erythema The final visual evaluation is conducted on the afternoon of the final day.

The 0-6 grading scale shown in Table 6 is used to assess the test sites for dryness and erythema. To maintain the evaluator's blindness to product assignment, visual assessments are conducted in a separate area away from the product application area.

TABLE 6
Erythema and Dryness grading scale.

| Grade | Erythema | Dryness |
|---|---|---|
| 0 | None | None |
| 1.0 | Barely | Patches of slight powderiness and redness |
| | perceptible | occasional patches of small scales may be |
| | | seen. Distribution generalized. |
| 2.0 | Slight redness | Generalized slight powderiness. Early |
| | | cracking or occasional small lifting scales |
| | | may be present |
| 3.0 | Moderate | Generalized moderate powderiness and/or |
| | redness | heavy cracking and lifting scales. |
| 4.0 | Heavy or | Generalized heavy powderiness and/or |
| | substantial | redness heavy cracking and lifting scales |
| 5.0 | Extreme | Generalized high cracking and lifting scales. |
| | redness | Powderiness may be present but not |
| | | prominent. May see bleeding cracks. |
| 6.0 | Severe redness | Generalized severe cracking. Bleeding cracks. |
| | | Bleeding cracks may be present. Scales large, |
| | | may be beginning to disappear. |

Instrumental readings are taken on the first (baseline) and final day of the study.

Mildness of test product is calculated as 1/(mean change in dryness at end of the study).

In addition to visual evaluation, instrumental assessments of the treated sites will be conducted using an evaporimeter and skin conductance meter as described in the reference above.

ii) Patch Testing

48 hr continuous or 14 day cumulative insult patch test: In the 48 hr patch test 5-15% solution/slurry of the product is applied onto the upper arm/back of the subject using a standard cotton pad. Irritation response is recorded for up to 24 hrs after removal of the patch. In the 14 day cumulative test a 5-15% solution/slurry of the product is applied repeatedly every 24 hrs for 14 days. Irritation response is recorded for up to 24 hrs after removal of patch.

Mildness of test product is evaluated as 1/(mean erythema at 24 hr after final patch removal).

c. Moisturization Test:

Each outer, lower leg of a test subject will be divided into three sites, 2.5 by 2.5 inch squares (upper, middle and lower) for a total of 6 test sites per subject. One or two of the sites will be untreated and will be included in the randomization of products. A technician will treat the sites once or twice with the designated amount of test material for 10 seconds. Cleansing products will remain on the test sites for a maximum of 90 seconds. Sites will be rinsed for 30 seconds each (e.g. using a temperature of 35 C), ensuring that the test material from one site does not contaminate another site. After rinsing, the test sites are gently dried with a paper towel. The application consists of dosing with up to 5 different test materials on the designated sites, one material per test site, and one or two untreated sites. The study personnel will perform the following wash procedure:

Test Phase: Visual Evaluation

The scale as shown in Table 7 will be used to assess the test sites for dryness.

TABLE 7

| Grade | Dryness Scale | Erythema Scale |
|---|---|---|
| 0.0 | No dryness | No erythema |
| 0.5 | Perceptible dryness, fine white lines | |
| 1.0 | Fine dry lines, white powdery look and/or | Mild erythema |
| | some uplifting flakes, on less than 30% of | |
| | the test site | |
| 1.5 | More uniform flaking, covering 30-50% of | |
| | the test site | |
| 2.0 | Uniform, marked flaking covering more | Moderate |
| | than 50% of the test site area and/or | confluent |
| | isolated scaling | erythema |
| 2.5 | Slight to moderate scaling | |
| 3.0 | Moderate to severe scaling with some | Marked |
| | uplifting of the scales | erythema |
| 3.5 | Severe scaling and/or slight fissuring | |
| 4.0 | Severe scaling and severe fissuring | Deep erythema |

Baseline visual assessments will be made prior to the start of the product application phase and thereafter, immediately before each of the instrumental assessments, to evaluate skin dryness and erythema. One trained evaluator will conduct all visual evaluations during the product application phase. The evaluator will examine both lower legs with the aid of an illuminated magnifying lamp with a 3 diopter lens and a shadow-free circular cool white fluorescent light source.

Instrumental Assessment

All instrumental evaluations will be taken following a 30-minute acclimation period. The indoor humidity and temperature data will be recorded and included in the final report. Instrumental measurements may be taken at some or all of the following time points: 0, 1, 2, 4, 6, 8 and 24 hours after product application. Instruments to be used with this protocol include: ServoMed Evaporimeter with EP1 or EP2 probe, Corneometer CM820, the Skicon Skin Hygrometer with the MT-8C probe, and the Moisture Checker. The room temperature will be maintained at 680 to 77° F. and 30% to 40% Relative Humidity.

Moisturization is defined as mean change from baseline of (visual dryness or skin hydration).

d) Moisturizer Deposition Test:

Precondition the subject's skin (arms/legs) with non-moisturizer containing product for up to 2 days prior to testing. A baseline extraction is performed to estimate level of moisturizer (e.g.: fatty acids) present on the skin prior to product application. Controlled single application of product to skin (arms or legs) is made. For wash, bar is rubbed on skin for 30 sec. and the lather left on for 90 sec., rinsed for 30 sec. (e.g. using a temperature of 35 C) then gently pat dry. Following this, the site is extracted using a suitable solvent (IPA)/methanol 1:1). The extraction is performed as follows: A glass cup (3 cm diameter) is placed on the skin. 3 mls of solvent is placed into this and gently stirred with a glass rod for 2 minutes. The solvent is removed with a pipette. This step is repeated with a fresh 3 mls of solvent, to collect a total of 6 mls extract. The extracts are analyzed for stearic acid/palmitic acid content using either LC/MS or GC/MS, or the like.

e) Skin Abrasiveness Test

Skin abrasiveness is defined as consumer rated response of abrasivity on a 0-9 scale (0 means no abrasion, 10 is abrasivity caused by a pouf (i.e. a showering implement composed of thin plastic filaments, see also e.g. U.S. Pat. No. 5,650,384 to Gordon et al.).

This test is performed with 50 untrained consumers. They are asked to rate the abrasiveness of the test product on a 0-9 point scale. The data is normalized based on their response to a bar with no exfoliants which is assigned a value of zero and a pouf that is assigned a value of 9. The test products are applied to the flex area of the forearm by wetting the bar and rubbing back and forth 10-15 times.

f) Cleansing Efficacy Test

Model dirt (sebum/makeup—e.g. lipstick or mascara) is applied to a designated area on the forearm/face. The site is washed with the product. For wash, the bar is rubbed on skin for 1 minute, rinsed for 30 secs (e.g. using a temperature of 35 C), and gently pat dry. Amount of soil/makeup removed is estimated from the difference in the chromammeter readings using e.g. a Minolta Chromameter®, Model CM 2002 taken before and after wash. Alternately, high magnification digital mages are collected and analyzed using Optimas® software to quantitate the amount of soil/makeup removed during the wash.

Make Up Application:

Makeup will be applied to the 3.5×2.5 cm marked area on the inner side of the forearms in the manner consistent with its normal use. Cosmetic products are to be applied in a standardized way to ensure that approximately equal weights of make-up are transferred and that coverage of the test area is uniform. The application standards for the makeup are:
Liquid make-up—20 μl pipette to the site and spread uniformly with gloved index finger.
Lipstick-Three overlapping swipes.
Eye Color Stick-Three overlapping swipes.
Mascara-spread uniformly using spatula for even coverage.

Soil Application:

Soils will be applied to the 3.5×2.5 cm marked area on the inner side of the forearms in the manner described below and is specific to each individual study if soils are being used. The application techniques for the soils are:
Grease—0.25 g-1.5 g. will be applied.
Food—0.25 g-1.5 g. will be applied.
Protein—0.25 g-1.5 g. will be applied.

Product Testing:

Baseline measurements will be performed using the Minolta Chromameter CM-2002. Make-up or Soil will then be applied to the delineated test sites as described above. Chromameter measurements will be taken again after the make up has dried for 10 minutes, then the make-up/soil will be removed. The standard washing procedure used to remove the make-up/soil is a 30-second wash with 0.5 cc of a liquid product with a 15-second rinse under running water using a suitable constant temperature (e.g. 35 C). When a towelette product is being used, the towelette is rubbed over the test site in a circular motion for 15 seconds. Final Chromameter measurement will be taken after the make-up/soil has been removed. This procedure may be performed twice a day for a period of up to 3 days. In repeat application studies visual assessments will be made for dryness and erythema using the standard visual grading scale as described above.

g) Skin Smoothness

Skin smoothness is evaluated (clinically) via Primos® (in-vivo optical skin topography measuring device supplied by GFM Esstezhnik GmbH, Berlin, Germany). Baseline roughness is measured (on leg/arms—starting dryness around grade 1-2). For wash, bar rubbed on skin for 30 secs and the lather left on for 90 secs, rinsed for 30 secs at 35 C. Measure again the roughness 30 minutes after wash process. This procedure may be performed twice a day for a period of up to 5 days.

Smoothness is defined as the mean decrease in roughness at end of study period. Alternately skin smoothness can also be evaluated in a consumer test as follows:

The consumer test protocol consists of:

1) Recruiting approx. 10-20 women in the age group of 25-65 and who are complexion bar users.

2) Use test and comparative products for a week each. Half the panelists would use the test product first and the other half would use the comparative product first.

1) At the end of the test, the panelists rate their preference (on a 0-5 point scale) on the attribute of “Skin feels smoother”. Smoothness is defined as the consumer rating on the 0-5 point scale.

h) Skin Softness

Skin softness may be evaluated using the Linear Skin Rheometer (Goodyear Scientific Instruments, UK). Exfoliated skin has less dry flakes—hence is more soft/less stiff. The test involves baseline skin rheometer readings (on the leg/arms) to measure the dynamic spring constant (mgf/mm) of skin which is related to skin stiffness/softness. For wash, the bar is rubbed on the skin for 30 secs and the lather left on for 90 secs, rinsed for 30 secs (at a suitable temperature e.g. 35 C), and the skin is gently pat dry. Next measure skin stiffness/softness 30 minutes after wash. This procedure may be performed twice a day for a period of up to 5 days. Softness is defined as the mean decrease in dynamic spring constant during the study period observed during the study period.

Alternately skin softness can also be evaluated in a consumer test as follows:

The test protocol consists of

1) Recruiting approx. 10-20 women in the age group of 25-65 and who are complexion bar users.

2) Use test and comparative products for a week each. Half the panelists would use the test product first and the other half would use the comparative product first.

3) At the end of the test, the panelists rate their preference (on a 0-5 point scale) on the attribute of “Skin feels softer”.

Softness is defined as the consumer rating on the 0-5 point scale.

i) pH Test Method

Form an aqueous slurry by blending 10 grams of the bar formula with 90 g of water to create a 10% slurry. The pH of the slurry is then measured at 25 C.

j) Zein Test Method

The cleansing base of the inventive toilet bar preferably have zein solubilities of under about 50, 40, 30, and most preferably under about 25 using the zein solubility method set forth below. The lower the zein score, the milder the product is considered to be. This method involves measuring the solubility of zein (corn protein) in cleansing base solutions as follows:

0.3 g of cleansing base and 29.7 g of water are mixed thoroughly. To this is added 1.5 g of zein, and mixed for 1 hour. The mixture is then centrifuged for 30 minutes at 3000 rpm. After centrifugation, the pellet is extracted, washed with water, and dried in a vacuum oven for 24 hours until substantially all the water has evaporated. The weight of the dried pellet is measured and percent zein solubilized is calculated using the following equation:
% Zein solubilized=100 (1-weight of dried pellet/1.5).

The % Zein is further described in the following references: E. Gotte, Skin compatibility of tensides measured by their capacity for dissolving zein protein, Proc. IV International Congress of Surface Active Substances, Brussels, 1964, pp 83-90.

h) Bar Sensory Exfoliation Index

The bar sensory exfoliation index is determined using the following procedure: The user takes the bar in one hand and rotates it under running water at 35 C. The number of rotations required for the exfoliant to be perceived (i.e. by tactile sensation) by the user is recorded. The bar exfoliation index is defined as the mean number of rotations required to perceive the exfoliant particles in the bar.

i) General Consumer Test Protocol

The test protocol consists of

1) Recruiting approx. 10-20 women in the age group of 25-65 and who are complexion bar users.

2) Use test and comparative products for a week each. Half the panelists would use the test product first and the other half would use the comparative product first.

2) At the end of the test, the panelists rate their preference on a 0-5 point scale for the following attributes:
Exfoliates
Provides Gentle Exfoliation
Moisturizes and exfoliates
Skin feels softer
Skin feels smoother
Is good for dry skin.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

1. A skin care or cleansing product, comprising:
(a) about 2 to 90% by wt. of an anionic surfactant;
(b) about 0.1 to 40% by wt. of an oil seed product wherein at least 50% by wt. of the product has a particle size range of about 0.01 to about 200 microns; and
(c) optionally a safe and effective amount of at least one active agent, moisturizing agent or blend thereof.

2. The product of claim 1 wherein the product is selected from a toilet bar, a cream, a lotion, a shampoo, a liquid soap, an isotropic structured shower gel, or a crystalline structured body wash.

3. A toilet bar, comprising:
(a) about 0 to 30% by wt. of a fatty acid soap;
(b) about 15 to 60% by wt. of a non-soap anionic surfactant; and
(c) about 0.01 to 15% by wt. of an oil seed product having a particle size diameter of about 0.1 to 70 microns.

4. The toilet bar of claim 3 which contains about 0 to 20% by wt. of a fatty acid soap and about 20 to 55% by wt. of a non-soap anionic surfactant.

5. The toilet bar of claim 3 further comprising a safe and effective amount of at least one active agent, moisturizing agent or blend thereof.

6. The toilet bar of claim 3 wherein the oil seed product is selected from flax, soybean, sunflower, rapeseed, or palm.

7. The toilet bar of claim 3 wherein the fatty acid soaps include a blend of C6 to C22 soaps.

8. The toilet bar of claim 3 wherein the non-soap anionic surfactant is selected from C8 to C14 acyl isethionates; C8 to C14 alkyl sulfates, C8 to C14 alkyl sulfosuccinates, C8 to C14 alkyl sulfonates; C8 to C14 fatty acid ester sulfonates, derivatives, and blends thereof.

9. A toilet bar, comprising:
(a) about 30 to 80% by wt. of a fatty acid soap;
(b) about 5 to 40% by wt. of a non-soap anionic surfactant; and
(c) about 0.01 to 15% by wt. of an oil seed product having a particle size diameter of about 0.1 to 70 microns.

10. The toilet bar of claim 9 which contains about 40 to 70% by wt. of a fatty acid soap and about 7 to 30% by wt. of a non-soap anionic surfactant.

11. The toilet bar of claim 9 further comprising a safe and effective amount of at least one active agent, moisturizing agent or blend thereof.

12. The toilet bar of claim 9 wherein the oil seed product is selected from flax, soybean, sunflower, rapeseed, or palm.

13. The toilet bar of claim 9 wherein the fatty acid soaps include a blend of C6 to C22 soaps.

14. The toilet bar of claim 9 wherein the non-soap anionic surfactant is selected from C8 to C14 acyl isethionates; C8 to C14 alkyl sulfates, C8 to C14 alkyl sulfosuccinates, C8 to C14 alkyl sulfonates; C8 to C14 fatty acid ester sulfonates, derivatives, or blends thereof.

15. A toilet bar, comprising:
(a) about 30 to 80% by wt. of a fatty acid soap;
(b) about 0 to 10% by wt. of a non-soap anionic surfactant; and
(c) about 0.01 to 15% by wt. of an oil seed product having a particle size diameter of about 0.1 to 70 microns.

16. The toilet bar of claim 15 which contains about 50 to 80% by wt. of a fatty acid soap and about 0 to 7% of a non-soap anionic surfactant.

17. The toilet bar of claim 15 further comprising a safe and effective amount of at least one active agent, moisturizing agent or blend thereof.

18. The toilet bar of claim 15 wherein the oil seed product is selected from flax, soybean, sunflower, rapeseed or palm.

19. The toilet bar of claim 15 wherein the fatty acid soaps include a blend of C6 to C22 soaps.

20. The toilet bar of claim 15 wherein the non-soap anionic surfactant is selected from C8 to C14 acyl isethionates; C8 to C14 alkyl sulfates, C8 to C14 alkyl sulfosuccinates, C8 to C14 alkyl sulfonates; C8 to C14 fatty acid ester sulfonates, derivatives, or blends thereof.
